(19) United States
(12) Patent Application Publication
(10) Pub. No.: US 2024/0070507 A1
(43) Pub. Date: Feb. 29, 2024

(54) METHOD FOR CREATING AND SYNCHRONIZING A NEURAL LINK BETWEEN A QUANTUM COMPUTER SYSTEM AND A IN VIVO NEURAL NETWORKS BY ENTANGLEMENT WITH QUANTUM DOTS VIA OPTOGENETICS

(71) Applicant: André Augusto CEBALLOS MELO, São Paulo (BR)

(72) Inventor: André Augusto CEBALLOS MELO, São Paulo (BR)

(21) Appl. No.: 17/894,409

(22) Filed: Aug. 24, 2022

(51) Int. Cl. G06N 10/40; A61N 5/06

(52) U.S. Cl. CPC G06N 10/40; A61N 5/0601; A61N 2005/0626

(57) ABSTRACT

A method for creating and synchronizing a neural link between a quantum computer system and in vivo neural networks by entanglement with quantum dots via optogenetics to measure the spins of two ions trapped in various directions, through quantum neural entanglement devices in addition to the use to synchronize the activity of the cerebral cortex and the reticular formation with a computational system and with the use of photosensitive semiconductor detectors to detect the semiconductor quantum dots that emit entangled polarizing photons, thus obtaining a neural network map that can present several important information, performed through semiconductor photon detector equipment and computer systems.

### BACKGROUND

Quantum entanglement emerged as a pivotal concept in a variety of fields, such as quantum computation,1 -communication,2-4 and -metrology.

Semiconductor quantum dots are capable of emitting polarization entangled photon pairs with ultralow multipair emission probability even at maximum brightness.

The teleportation of the entanglement is enabled by two-photon interference to perform a so-called Bell state measurement (BSM). The success of a BSM strongly depends on the photon indistinguishability, which, in turn, depends on the photon sources and can be experimentally accessed by probing the interference visibility in a Hong-Ou-Mandel (HOM) experiment.

Furthermore, these systems are limited in range by the fundamental laws of quantum mechanics.

Optogenetics is a relatively new technique in the neurobiology field that integrates the use of light (optics) and genetic engineering to manipulate neural activity. It broadly involves transfecting neurons with light-sensitive proteins (opsins) that, depending on the type of opsin used, can selectively activate or silence those neurons using pulses of visible light.

It has now taken hold in the neuroscience field and revolutionized how we study the brain. Prior to its application, it was challenging for scientists to discriminate the neural pathways involved in certain physiological responses and behaviors. Alternative methods such as pharmacological or electrical stimulation lack the same temporal and anatomical precision that can be achieved through optogenetic tools.

With the rapid development of related technologies, optogenetics has opened the door to therapeutic applications such as deep brain stimulation for the treatment of Parkinson's disease, prevention of seizures for epilepsy, and even in the pacing of heart muscle for cardiac dysfunction.

Optogenetics offers a highly targeted approach to neural modulation from the scale of individual neurons in living tissue, to freely moving animals—making it a powerful tool for any neuroscience researcher.

Although a wide range of technologies now fall under the optogenetics, generally they can be divided into two main categories; the tools used to express opsins in the target tissue, and those used to stimulate (activate/deactivate cells) and measure the resulting response.

Tools and techniques used for opsin expression and stimulation for in vivo studies, as use of Opsin, Quantum Dots and others.

Opsin expression in the target tissue. Because microbial opsins aren't naturally expressed in mammalian tissue, the target cells must be genetically engineered to express these proteins.

Several techniques are available for introducing opsins into live cells, including DNA transfection, electroporation, and transgenic expression systems. In the brain, viral-mediated gene delivery has become the method of choice for most neuroscientists due to its ability to target different regions of brain tissue with increased accuracy.

The second major factor that needs to be considered for optogenetic studies are the tools required to stimulate the opsin expressing cells and measure the resulting response. Most light targeting devices available for in vivo animal studies involve the use of a tethered or wireless head-mounted system, with the addition of a secondary piece of equipment for recording the physiological response.

While tethered and head-mounted systems have improved dramatically since the initial application of optogenetics, they still have their limitations, including: • Increased susceptibility to infection at interface site; • Impact on normal physiology and behavior (due to tethering or bulky headpiece); • Restriction of experimental duration and protocols due to restraint and/or limited battery-life; and • Additional costs of tether maintenance and replacement some references related to the matter presented here can be found in the state of the art, which we highlight below:
J. L. O'Brien, A. Furusawa, and J. Vučković, “Photonic quantum technologies,” Nat. Photonics 3, 687-695 (2009). https://doi.org/10.1038/nphoton.2009.229, Google ScholarCrossref, ISI;
N. Gisin and R. Thew, “Quantum Communication,” Nat. Photonics 1, 165-171 (2007). https://doi.org/10.1038/nphoton.2007.22, Google ScholarCrossref, ISI;
H. J. Kimble, “The quantum internet,” Nature 453, 1023-1030 (2008). https://doi.org/10.1038/nature07127, Google ScholarCrossref, ISI;
A. K. Ekert, “Quantum cryptography based on Bell's theorem,” Phys. Rev. Lett. 67, 661-663 (1991). https://doi.org/10.1103/PhysRevLett.67.661, Google ScholarCrossref;
C. L. Degen, F. Reinhard, and P. Cappellaro, “Quantum sensing,” Rev. Mod. Phys. 89, 035002 (2017). Google ScholarCrossref;
A. Lenhard, J. Brito, S. Kucera, M. Bock, J. Eschner, and C. Becher, “Single telecom photon heralding by wavelength multiplexing in an optical fiber,” Appl. Phys. B 122, 20 (2016). https://doi.org/10.1007/s00340-015-6284-9, Google ScholarCrossref;
Alferov, Zh.I. The history and future of semiconductor heterostructures. Semiconductors 1998, 32, 1-14, DOI: 10.1134/1.1187350;
2shoori, R. C. Electrons in artificial atoms. Nature 1996, 379, 413-419, DOI: 10.1038/379413a0;
Kubendhiran, S.; Bao, Z.; Dave, K.; Liu, R.-S. Microfluidic Synthesis of Semiconducting Colloidal Quantum Dots and Their Applications. ACS Appl. Nano Mater. 2019, 2, 1773-1790, DOI: 10.1021/acsanm.9b00456;
Shirasaki, Y.; Supran, G. J.; Bawendi, M. G.; Bulovic, V. Emergence of colloidal quantum-dot light-emitting technologies. Nat. Photonics 2013, 7, 13-23, DOI: 10.1038/nphoton.2012.328;
The patent document WO2022133501, WIRELESS RECORDING SYSTEM-ON-CHIP FOR DISTRIBUTED NEURAL INTERFACE SYSTEMS WITH INDUCTIVE POWER DELIVERY AND UWB DATA TRANSMISSION. Systems and method for wireless recording system-on-chips for distributed neural interface systems with inductive power delivery and UWB data transmission are described. In an embodiment, the system includes an implantable neural interface including: an electrode array having several electrodes; front end circuitry including: one or more digital components, and at least one amplifier coupled to a first electrode and a second electrode of the electrode array, wherein the amplifier and the first electrode and the second electrode form a sensing channel configured to sense electrical activity; and a transceiver including: several digital components; a power harvesting system that receives RF energy through a wireless power link; and a wireless clock receiver that provides a clock signal to the one or more digital components of the front end circuitry and the several digital components of the transceiver;
The patent document US2022198146, SYSTEM AND METHOD FOR END-TO-END NEURAL ENTITY LINKING. Various methods, apparatuses/systems, and media for end-to-end entity linking are disclosed. The system includes a processor; and a memory operatively connected to the processor via a communication interface, the memory storing computer readable instructions, when executed, causes the processor to: detect all named entity mentions from a plurality of data sources; compute, in response to detecting, entity embeddings in a knowledge graph by implementing context information and a margin-based loss function; validate the entity embeddings; deploy, in response to validating the entity embeddings, a machine learning model to match character and semantic information, respectively; and link, in response to deployment of the wide and deep learning model, the named mentions in text with corresponding entities in the knowledge graph;
The patent document WO2022116207, CODING METHOD, DECODING METHOD, CODING APPARATUS, AND DECODING APPARATUS. The present application provides a coding method, a decoding method, a coding apparatus, and a decoding apparatus. The coding method comprises coding, by using a neural network-based coding technology, an image to be coded to obtain a code stream of said image; transmitting the code stream of said image by means of a first communication link; and transmitting, by means of a second communication link, model parameters of a neural network model comprised in the neural network-based coding technology. According to the solution provided by the present application, the management and transmission problems of model parameters of neural network models can be solved well, and the bit consumption of code streams is reduced; in addition, challenges of low-latency transmission caused by the presence of the management and transmission requirements of model parameters can be reduced;
The patent document WO2022108206, METHOD AND APPARATUS FOR COMPLETING DESCRIBABLE KNOWLEDGE GRAPH. Disclosed are a method and apparatus for completing a describable knowledge graph. According to the present invention, provided is the apparatus for completing a describable knowledge graph, comprising: a processor; and a memory connected to the processor, wherein the memory stores program instructions executable by the processor to: extract a plurality of relation paths for connecting a subject and an object in a query triple including the subject, a predicate, and the object; generate a plurality of describable segments by using the extracted plurality of relation paths; extract an embedding vector for each of the generated plurality of describable segments by using a neural network model in which a CNN and an LSTM are combined; compare the semantic similarity between the plurality of describable segments represented by the embedding vector and the query predicate included in the query triple, by using an attention mechanism; and determine a segment having high importance for link prediction regarding the query triple from among the plurality of describable segments via the semantic similarity comparison; and
The patent document US2022121907 Organizing Neural Networks Methods, systems, and apparatus, including computer programs encoded on a computer storage medium, for organizing trained and untrained neural networks. In one aspect, a neural network device includes a collection of node assemblies interconnected by between-assembly links, each node assembly itself comprising a network of nodes interconnected by a plurality of within-assembly links, wherein each of the between-assembly links and the within-assembly links have an associated weight, each weight embodying a strength of connection between the nodes joined by the associated link, the nodes within each assembly being more likely to be connected to other nodes within that assembly than to be connected to nodes within others of the node assemblies.

### SUMMARY

A method for creating and synchronizing a neural link between a quantum computer system and in vivo neural networks by entanglement with quantum dots via optogenetics to measure the spins of two ions trapped in various directions, through quantum neural entanglement devices in addition to the use to synchronize the activity of the cerebral cortex and the reticular formation with a computational system and with the use of photosensitive semiconductor detectors to detect the semiconductor quantum dots that emit entangled polarizing photons, thus obtaining a neural network map that can present several important information, performed through semiconductor photon detector equipment and computer systems.

### DETAILED DESCRIPTION

The present disclosure overcomes the limitations of the prior art. With optogenetics now taking a shift towards therapeutic research applications, there is a need for light delivery/stimulation tools that are more suited to long-term studies.

As such, wireless telemetry-based systems are becoming increasingly popular for in vivo animal research, as they facilitate long-term, unrestrained experimentation on animals in their home environment.

Fully implantable wireless optogenetic solution to offer both optogenetic stimulation and recording (EEG, ECG, and EMG) within the same device. Designed for use in vivo is an ideal solution for researchers looking to apply extended optogenetic stimulation protocols and record quantitative biopotential data in long-term experiments without the restrictions of battery power and tethers.

Quantum Dots and Their Applications:

It has been many years since the first works on the reduced dimensionality of semiconductors, which led to the concept of “artificial atoms”, or quantum dots (QDs). These semiconductor nanocrystals, with nanometer-sized diameters, exhibit quantum size effects in their optical and electronic properties. In particular, tunable and efficient photoluminescence (PL), with narrow emission and photochemical stability, and core-shell structures are commonly achieved today for many QD materials systems. As a consequence, QDs have been incorporated as active elements in a wide variety of devices and applications. Many of these applications are now commercially available and are incorporated into our daily life, such as the case of QD-based displays.

The high luminescence, narrow emission, and (depending on the elemental composition) low toxicity and biocompatibility of QDs make them a perfect candidate for bioimaging, diagnostics, and biosensing applications, mostly related to biomedical and environmental sciences.

Researchers at MIT have developed a method that lets them read the signal from fluorescent sensors that are as deep as 5.5 centimeters in tissue. Previously, it was very difficult to get a good signal from a fluorescent sensor placed that deep, as fluorescence emitted by the tissue itself would muddy the signal. The new technique involves using a laser with an oscillating wavelength to illuminate the fluorescent sensor, resulting in the emitted fluorescence to double its frequency. This allows the researchers to easily pick out the fluorescence signal from the background noise.

This new approach hopes to increase the clinical utility of fluorescence.

Perhaps the most famously weird feature of quantum mechanics is nonlocality: Measure one particle in an entangled pair whose partner is miles away, and the measurement seems to rip through the intervening space to instantaneously affect its partner. This “spooky action at a distance” (as Albert Einstein called it) has been the main focus of tests of quantum theory.

Contextuality says that properties of particles, such as their position or polarization, exist only within the context of a measurement. Instead of thinking of particles' properties as having fixed values, consider them more like words in language, whose meanings can change depending on the context.

Investigating these links could help guide researchers in developing new quantum computing approaches and algorithms.

And with renewed theoretical interest comes a renewed experimental effort to prove that our world is indeed contextual. Cabello, in collaboration with Kihwan Kim at Tsinghua University in Beijing, China, published a paper in which they claimed to have performed the first loophole-free experimental test of contextuality.

The Northern Irish physicist John Stewart Bell is widely credited with showing that quantum systems can be nonlocal. By comparing the outcomes of measurements of two entangled particles, he showed with his eponymous theorem of 1965 that the high degree of correlations between the particles can't possibly be explained in terms of local “hidden variables” defining each one's separate properties. The information contained in the entangled pair must be shared nonlocally between the particles.

John Bell, Simon Kochen and Ernst Specker (from top) proved a theorem in the late 1960s showing that quantum systems cannot possibly have fixed values for all properties in all contexts.

Bell also proved a similar theorem about contextuality. He and, separately, Simon Kochen and Ernst Specker showed that it is impossible for a quantum system to have hidden variables that define the values of all their properties in all possible contexts.

In Kochen and Specker's version of the proof, they considered a single particle with a quantum property called spin, which has both a magnitude and a direction. Measuring the spin's magnitude along any direction always results in one of two outcomes: 1 or 0.

The researchers then asked: Is it possible that the particle secretly “knows” what the result of every possible measurement will be before it is measured? In other words, could they assign a fixed value—a hidden variable—to all outcomes of all possible measurements at once?

Quantum theory says that the magnitudes of the spins along three perpendicular directions must obey the “101 rule”: The outcomes of two of the measurements must be 1 and the other must be 0. Kochen and Specker used this rule to arrive at a contradiction. First, they assumed that each particle had a fixed, intrinsic value for each direction of spin. They then conducted a hypothetical spin measurement along some unique direction, assigning either 0 or 1 to the outcome. They then repeatedly rotated the direction of their hypothetical measurement and measured again, each time either freely assigning a value to the outcome or deducing what the value must be in order to satisfy the 101 rule together with directions they had previously considered.

They continued until, in the 117th direction, the contradiction cropped up. While they had previously assigned a value of 0 to the spin along this direction, the 101 rule was now dictating that the spin must be 1. The outcome of a measurement could not possibly return both 0 and 1. So the physicists concluded that there is no way a particle can have fixed hidden variables that remain the same regardless of context.

In 2009, contextuality, got a direct application: One of the simplified versions of the original Bell-Kochen-Specker theorem was shown to be equivalent to a basic quantum computation.

The proof, named Mermin's star after its originator, David Mermin, considered various combinations of contextual measurements that could be made on three entangled quantum bits, or qubits. The logic of how earlier measurements shape the outcomes of later measurements has become the basis for an approach called measurement-based quantum computing. The discovery suggested that contextuality might be key to why quantum computers can solve certain problems faster than classical computers—an advantage that researchers have struggled mightily to understand.

The experiment entailed measuring the spins of two entangled trapped ions in various directions, where the choice of measurement on one ion defined the context for the other ion.

The physicists showed that, although making a measurement on one ion does not physically affect the other, it changes the context and hence the outcome of the second ion's measurement.

Examples of quantum neural entanglement devices include, but are not limited to, as example of a potential targets.

In addition to the use to synchronize the activity of the cerebral cortex with a computer system, a second target would be the Reticular formation

The ascending reticular activating system (ARAS), also known as the extrathalamic control modulatory system or simply the reticular activating system (RAS), is a set of connected nuclei in the brains of vertebrates that is responsible for regulating wakefulness and sleep-wake transitions. The ARAS is a part of the reticular formation and is mostly composed of various nuclei in the thalamus and a number of dopaminergic, noradrenergic, serotonergic, histaminergic, cholinergic, and glutamatergic brain nuclei.

The Reticular Activating System is responsible for our wakefulness, our ability to focus, our fight-flight response, and how we ultimately perceive the world

The reticular formation is a set of interconnected nuclei that are located throughout the brainstem. It is not anatomically well defined, because it includes neurons located in different parts of the brain. The neurons of the reticular formation make up a complex set of networks in the core of the brainstem that extends from the upper part of the midbrain to the lower part of the medulla oblongata. The reticular formation includes ascending pathways to the cortex in the ascending reticular activating system (ARAS) and descending pathways to the spinal cord via the reticulospinal tracts.

Neurons of the reticular formation, particularly those of the ascending reticular activating system, play a crucial role in maintaining behavioral arousal and consciousness. The overall functions of the reticular formation are modulatory and premotor, involving somatic motor control, cardiovascular control, pain modulation, sleep and consciousness, and habituation. The modulatory functions are primarily found in the rostral sector of the reticular formation and the premotor functions are localized in the neurons in more caudal regions.

The reticular formation is divided into three columns: raphe nuclei (median), gigantocellular reticular nuclei (medial zone), and parvocellular reticular nuclei (lateral zone). The raphe nuclei are the place of synthesis of the neurotransmitter serotonin, which plays an important role in mood regulation. The gigantocellular nuclei are involved in motor coordination. The parvocellular nuclei regulate exhalation.

The reticular formation is essential for governing some of the basic functions of higher organisms and is one of the phylogenetically oldest portions of the brain.

The method proposed here Method for Creating and synchronizing a neural link between a quantum computer system and a in vivo neural networks by entanglement with quantum dots via Optogenetics, proposes to measure the spins of two ions trapped in various directions, through quantum neural entanglement devices in addition to the use to synchronize the activity of the cerebral cortex and the reticular formation with a computational system and with the use of photosensitive semiconductor detectors to detect the semiconductor quantum dots that emit entangled polarizing photons, thus obtaining a neural network map that can present several important information. Performed through at east by semiconductor photon detector equipment and computer systems.

We claim:

1. A method for creating and synchronizing a neural link between a quantum computer system and in vivo neural networks by entanglement with quantum dots via optogenetics, comprising:
measuring spins of at least two ions trapped in various directions, through quantum neural entanglement devices, in addition to use to synchronize activity of the cerebral cortex and a reticular formation with a computational system, and using at least photosensitive semiconductor detectors to detect the semiconductor quantum dots that emit entangled polarizing photons, obtaining a neural network map that can presents information, performed at least by semiconductor photon detector equipment and computer systems.
